(19) United States
(12) Patent Application Publication
(10) Pub. No.: US 2011/0275807 A1
(43) Pub. Date: Nov. 10, 2011

(54) Application of Evodiamine in preparing medicaments for Alzheimer's disease

(75) Inventors: Lianfeng Zhang, Beijing (CN); Chuan Qin, Beijing (CN); Shumin Yuan, Beijing (CN); Shumei Wang, Beijing (CN)

(73) Assignee: Institute of Laboratory Animal Science, Chinese Academy of Medical Sciences

(21) Appl. No.: 12/883,194

(22) Filed: Sep. 16, 2010

(30) Foreign Application Priority Data
May 5, 2010 (CN) 201010162103.X

(51) Int. Cl. C07D 487/14

(52) U.S. Cl. 544/245

(57) ABSTRACT

A method of preparing medicaments for Alzheimer's disease includes the step of administering Evodiamine. Evodiamine can improve the main symptom of Alzheimer's disease, namely, the loss of learning and memory abilities, and delay the progress of Alzheimer's disease. The effects of Evodiamine are represented by following two aspects. Firstly, it can inhibit the over-activation of the microglias in the brain tissue, consistent with the reduction of the over-expression of the cytokines IL-Iβ, IL-6 and TNF-α. Secondly, it can reduce the expression of the cyclaoxygenase-2 thus inhibiting the inflammation in the central nervous system. Researches on curative effects and molecular mechanism of applying Evodiamine to Alzheimer's disease have proved that Evodiamine has a good therapeutic effect and has a potential to develop into a clinical therapy medicament.

### BACKGROUND OF THE PRESENT INVENTION

1. Field of Invention

The present invention relates to a medicine field, and more particularly to an application of Evodiamine in preparing medicaments for Alzheimer's disease.

2. Description of Related Arts

Alzheimer's disease (AD) is a chronic and progressive neurodegenerative disease. It is mainly represented as the cognitive impairment, the formation of senile plaques in brain tissue, the neurofibrillary tangles accompanied by the neuronal loss.

Evodiamine, having a molecular formula of C19H17N3O, exists in the fruit of Evodia rutaecarpa of the Rutaceae plant, and its structure and related biological activity have been reported in the published literatures. However, no report on an application of Evodiamine in preparing medicaments for Alzheimer's disease has been seen.

### SUMMARY OF THE PRESENT INVENTION

An object of the present invention is to provide an application of Evodiamine in preparing medicaments for Alzheimer's disease.

Accordingly, in order to accomplish the above object, the present invention provides a method of preparing medicaments for Alzheimer's disease comprising the step of administering Evodiamine.

Evodiamine can improve the main symptom of Alzheimer's disease (AD), namely, the loss of learning and memory abilities, and delay the progress of Alzheimer's disease. The effects of Evodiamine are represented by following two aspects. Firstly, Evodiamine can inhibit the over-activation of the microglias in the brain tissue, and reduce the over-expression of the cytokines IL-Iβ, IL-6 and TNF-α. Secondly, Evodiamine can reduce the expression of the cyclaoxygenase-2, thus inhibiting the inflammation in the central nervous system.

Evodiamine is an Evodiamine monomer or a mixture containing an Evodiamine monomer.

By the APPswe/PSΔE9 transgenic model of Alzheimer's disease built in the lab, researches on the curative effect and molecular mechanism of applying Evodiamine to Alzheimer's disease have proved that Evodiamine has a good therapeutic effect and has a potential to develop into a clinical therapy medicament.

These and other objectives, features, and advantages of the present invention will become apparent from the following detailed description, the accompanying drawings, and the appended claims.

### DETAILED DESCRIPTION OF THE PREFERRED EMBODIMENT

The present invention is further explained with detailed embodiments as follows.

### Example 1

Evodiamine treatment improves learning and memory abilities of the model group mice with Alzheimer's disease.

The evodiamine treatment group was administrated in the standard diet plus evodiamine in a dose of 100 mg/kg body weight/day for 3 months. The model group received a standard diet as the placebo control. After a three-month continuous administration, the Morris water-maze tests were performed to test the effect of evodiamine on improvement of learning and memory abilities of the Alzheimer's disease mice model. The result shows, compared with the model group mice, the learning and memory abilities of are significantly improved by the treatment of evodiamine. Therefore, it is proved that the Evodiamine treatment can improve the learning and memory abilities of the mice with Alzheimer's disease.

### Example 2

Evodiamine treatment inhibits the over-expression of the brain tissue inflammation related cytokines of the model group mice with Alzheimer's disease.

The evodiamine treatment group was administrated in the standard diet plus evodiamine in a dose of 100 mg/kg body weight/day for 3 months. The model group received a standard diet as the placebo control. After a three-month continuous administration, it is verified by ELISA that, compared with the model group, the IL-Iβ, IL-6 and TNF-α in the brain tissues are significantly decreased by the treatment of evodiamine. Therefore, it is proved that Evodiamine treatment can inhibit the brain tissue inflammation of the mice with Alzheimer's disease.

### Example 3

Evodiamine treatment inhibits the expression of the cyclaoxygenase-2 (COX-2) of the model group mice with Alzheimer's disease.

The evodiamine treatment group was administrated in the standard diet plus evodiamine in a dose of 100 mg/kg body weight/day for 3 months. The model group received a standard diet as the placebo control. After a three-month continuous administration, it is verified by the Western Blotting that, compared with the model group, the expression of the cyclaoxygenase-2 (COX-2) in the brain tissue is reduced by the treatment of evodiamine Therefore, it is proved that evodiamine treatment can inhibit the inflammation in the central nervous system. The inflammation is one of main reasons resulting in the pathological development of Alzheimer's disease, so Evodiamine can treat Alzheimer's disease.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A method of preparing medicaments for Alzheimer's disease, comprising the step of administering Evodiamine.

2. The method, as recited in claim 1, wherein the Evodiamine is an Evodiamine monomer or a mixture containing an Evodiamine monomer.

3. A medicament for Alzheimer's disease comprising Evodiamine.

4. The medicament, as recited in claim 3, wherein said Evodiamine is an Evodiamine monomer or a mixture containing an Evodiamine monomer.
